(19) United States
(12) Patent Application Publication
(10) Pub. No.: US 2011/0069278 A1
(43) Pub. Date: Mar. 24, 2011

(54) MEDICAL HAND ATTACHMENT WITH LIGHTING

(75) Inventor: Volker Gueder, Heidelberg (DE)

(73) Assignee: Geuder AG, Heidelberg (DE)

(21) Appl. No.: 12/090,581

(22) PCT Filed: Oct. 16, 2007
(86) PCT No.: PCT/DE07/01838

§ 371 Date: Dec. 7, 2010

(30) Foreign Application Priority Data
Oct. 30, 2006 (DE) 10 2006 051 736.9

(51) Int. Cl. A61B 3/10; A61B 1/07

(52) U.S. Cl. 351/221

(57) ABSTRACT

A handheld medical instrument for illumination, in particular for application in ophthalmology, comprising a housing serving as a handle, a probe extending from the housing, and a light outlet at the end, is characterized by a light source integrated into the handheld instrument.

The invention relates to a handheld medical instrument for illumination, in particular for application in ophthalmology, comprising a housing serving as a handle, a probe extending from the housing, and a light outlet at the end.

Handheld medical instruments for illumination are known in a wide variety of embodiments from the art. If handheld instruments for application in ophthalmology are involved, an extremely small design is routinely required. This applies in particular to the probe extending from the housing and having a light outlet at the end. If the probe serves for being introduced into a human body, for example into the human eye, not only the small design but also unimpeded handling of the instrument is required.

Generic handheld medical instruments, in particular for application in ophthalmology, are extremely small in their construction. This can be attributed to the fact that the light source together with energy supply is situated outside the handheld instrument. The light from an external light source is regularly coupled to the handheld instrument via an optical fiber, the light being guided in the interior of the housing through the probe as far as the light outlet. Within the housing, the beam path can be defined as desired, for example via an optical fiber.

The generic handheld instruments known from the art are problematic, however, insofar as the connected optical waveguide makes them more difficult to handle or impedes them while they are being handled. Moreover, there is always the risk of the sensitive optical waveguide, usually comprising a shielded glass fiber, being bent and irreversibly damaged or destroyed in the process.

The present invention is based on the object, therefore, of configuring and developing a handheld medical instrument for illumination, in particular for application in ophthalmology, in such a way that unimpeded handling is possible in conjunction with an extremely simple construction.

The above object is achieved according to the invention by means of a handheld medical instrument having the features of patent claim 1. Accordingly, the generic handheld instrument is characterized by a light source integrated into the handheld instrument.

In a surprisingly simple manner, a departure is made from the prior art, which, in conjunction with a very small design, involves the use of an external light source whose light is coupled into the handheld instrument via an optical fiber. On the contrary, namely in a departure from the prior art, the light source is now integrated into the handheld instrument. This has the huge advantage that the use of an external optical fiber is obviated. Just by virtue of this measure, the costs of a corresponding handheld instrument can be very considerably reduced moreover, there is no risk of the external optical fiber being blinded, whereby it becomes necessary to replace the optical fiber—despite the state of the optical fiber otherwise.

In an advantageous manner, the probe is formed as a rectilinear or bent tube, which preferably comprises high-grade steel, titanium or a titanium alloy. The light is conducted through the housing and through the probe as far as the light outlet. In accordance with the positioning of the free end of the probe, it is possible to illuminate any desired regions of the body, in particular including body cavities or internally within the eye.

In an especially advantageous manner, the light source is embodied as a miniaturized light source, thereby permitting the integration of the light source into the handheld instrument. Since a light source fundamentally emits heat, it is furthermore advantageous if the light source is surrounded by a material suitable for heat dissipation or by a heat conducting device. Such a measure makes it possible to effectively avoid overheating within the handheld instrument.

In the context of a first variant, it is conceivable to arrange the light source in the housing. The light emitted by the light source is conducted to the light outlet via an optical fiber extending through the probe. In this case, it is advantageous if the light source of the probe is arranged, such that the light has to cover extremely small distances within the instrument.

In a furthermore advantageous manner, the light from the light source is coupled into the optical fiber via optical components. The optical components can be a lens or an arrangement of lenses. It is likewise conceivable for the optical components to comprise a filter or an arrangement of filters. The light, proceeding from the integrated light source, can thus be influenced, for example focused or parallelized, as desired. In accordance with the requirements, it is likewise conceivable to perform polarization of the light by means of suitable grating. Specific wavelength ranges can be filtered out or—conversely—conducted to the light outlet. Optimum adaptation to the respective demands is possible.

The optical fiber can be formed as a singular monofiber. It is likewise conceivable to embody the optical fiber as a fiber bundle. In principle, it is conceivable to use optical fibers that exclusively guide white light.

With regard to a protected arrangement of the optical fiber and with regard to the light emergence, it is conceivable for the optical fiber to end within the probe, preferably just before the free end thereof. The optical fiber can likewise terminate flush with the free end of the probe. Furthermore, it may be advantageous if the optical fiber ends with a flat or rounded end, within or outside the probe, depending on the illumination demands.

In an especially refined manner, the optical fiber is displaceable from a position with the free end within the probe into a position with the free end at least slightly outside the probe. Different illumination situations can thus be brought about, the optical fiber in the retracted position being reliably accommodated in the probe.

It should be noted at this point that, in the context of the requirement for one hundred percent tightness, the optical fiber is adhesively bonded or sealed within the probe. Mobility of the optical fiber is dispensed with in such a case. However, it would—alternatively—be conceivable in this case to realize quasi displaceability of the optical fiber by a relative movement of the probe with respect to the housing.

The displaceability of the optical fiber could be effected by means of an actuating member that is assigned to the handle and acts directly or indirectly on the optical fiber.

In the context of a second embodiment, the light source is not arranged within the housing, for instance, but rather in the probe, to be precise preferably in the vicinity of the light outlet. Such a configuration requires a very considerable degree of miniaturization of the light source.

In such a case, too, it is conceivable for optical components to be arranged between the light source and the light outlet, within the probe, and it is likewise necessary to miniaturize said optical components in the context of such a configuration. The optical components can comprise a lens or an arrangement of lenses. A filter or an arrangement of filters can likewise be provided.

Furthermore, it is conceivable for an optical assembly, which is preferably formed in the form of a miniaturized lens or a miniaturized lens arrangement, to be assigned directly to the light outlet. In this case, said lens or lens arrangement would define the light outlet and terminate the probe in the front region.

The lens or lens arrangement can terminate flush with the free end of the probe. It is likewise conceivable for the lens or lens arrangement to be arranged within the probe, preferably just before the free end thereof. The configuration of the lens or lens arrangement, in a furthermore advantageous manner, is such that it ends with a flat or rounded end within or outside the probe, depending on the illumination demands. The light beam can be concentrated or spread by means of different configurations of the lens or the lens arrangement and through corresponding positioning.

It is furthermore conceivable for the lens or lens arrangement to be displaceable from a position within the probe into a position at least slightly outside the probe, the displaceability of the lens or lens arrangement being effected by an actuating member assigned to the probe or to the housing.

It has already been mentioned above that the light source, in an especially advantageous manner, is a greatly miniaturized light source, namely in order alternatively to accommodate it in the housing or even in the probe. In the context of the miniaturization sought, a light emitting diode (LED) is especially suitable as a light source, a light emitting diode additionally having the advantage of emitting only extremely little heat, at any rate in comparison with a conventional light source.

The light source can be, in principle, a light emitting diode whose power suffices for illumination in the ophthalmology area. It is likewise conceivable for the light source to comprise two or more light emitting diodes (LEDs), in which case provision can be made of two or more light emitting diodes for emitting light of different wavelengths. The light emitting diodes can optionally be switched individually or jointly.

In a furthermore advantageous manner, the intensity of the light emitted by the light source or the intensity of the light that can be coupled into the optical fiber is adjustable, such adjustment being possible by means of the energy supply or by means of corresponding filters or alternatively by means of individual light emitting diodes being supplementary switched in or switched out.

In principle, it is conceivable for the light source to be connected to an external energy source or power source preferably via a power cable leading into the housing. Precisely with regard to unimpeded handling of the handheld instrument, it is advantageous if the light source comprises an internal energy source in the form of a battery or a rechargeable battery. The rechargeable battery could be able to be charged via a charging cable or inductively via a charging device, preferably in a charging station.

Particularly with regard to effortless cleaning of the handheld instrument, it is advantageous if the housing and the probe with the light source situated therein and, if appropriate, with the energy source situated therein are completely sealed off or encapsulated toward the outside. In the context of such a configuration, it is only necessary to clean the exterior of the instrument since, on account of the sealing, no contaminations whatsoever can pass into the probe and/or into the housing.

Alongside a single-part configuration of the housing in the fully encapsulated state, it is conceivable for the housing to be embodied in two parts and preferably to be able to be opened on the side remote from the light outlet. That part of the housing which encompasses the battery or the rechargeable battery could be configured in removable fashion, such that, in order to renew the battery or the rechargeable battery, it is only necessary to exchange the rear part of the housing, namely together with the battery situated therein or with the rechargeable battery situated therein.

In a particularly refined manner, the light source together with the energy supply, preferably in the form of a monolithic assembly, could be removable from the housing or be removable with a part of the housing. In the flanged state, the removable part of the housing would have to be fixed in sealing fashion with respect to the other part of the housing.

In a furthermore advantageous manner, assemblies comprising the light source, with light sources of different wavelengths and/or different light intensities, are provided for exchange purposes, such that this assembly can be exchanged as required for an assembly having other performance data.

It could likewise be advantageous for the housing to comprise a coupling location for coupling out light by means of an external optical fiber that can be coupled thereto. In the context of such a configuration, the handheld instrument can be used as a quasi light source for coupling into an optical fiber, namely for other or supplementary purposes of use.

The actuation of the handheld instrument, namely switching the light source on and off, can be effected in different ways. In principle, it is conceivable for the housing to be assigned a switch for switching the light source on and off, in which case said switch, in an especially advantageous manner, is situated under the outer skin of the housing, such that in this case, too, only contamination of the surface of the housing can take place. An encapsulation of the entire handheld instrument is advantageous.

There are various possibilities, then, for configuring and developing the teaching of the present invention in an advantageous manner. In this respect, reference should be made on the one hand to the patent claims subordinate to patent claim 1, and on the other hand to the following explanation of exemplary embodiments of the invention with reference to the drawing. In conjunction with the explanation of the preferred exemplary embodiments of the invention with reference to the drawing, in general preferred configurations and developments of the teaching are explained as well. In the drawing:

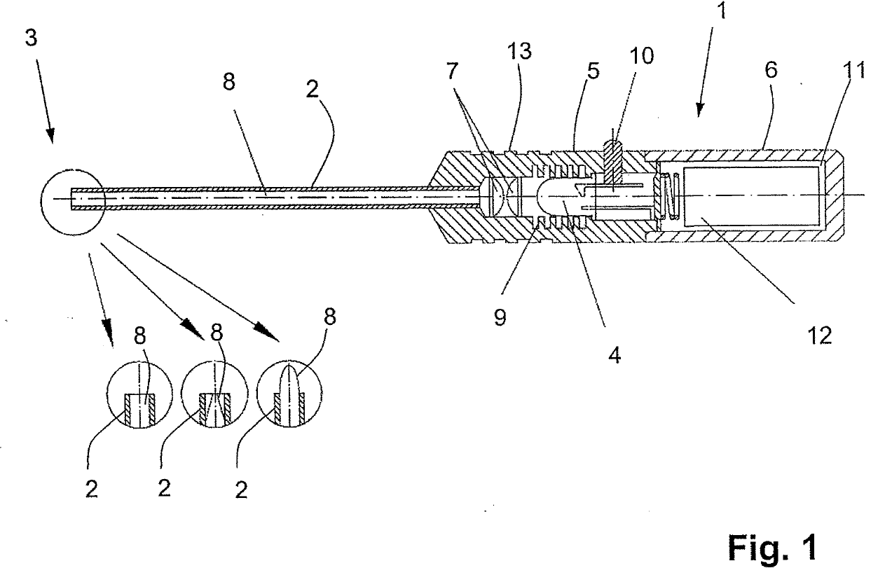

FIG. 1 shows in a schematic view, in section, a first exemplary embodiment of a handheld instrument according to the invention, wherein both the light source and the power supply are integrated into the housing;

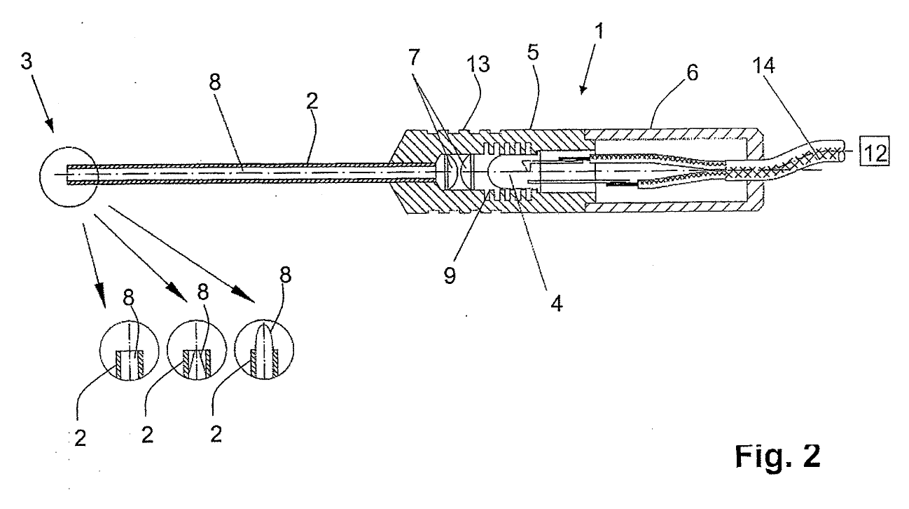

FIG. 2 shows in a schematic view, in section, a second exemplary embodiment of a handheld instrument according to the invention, in which the light source is integrated into the housing and the energy supply is effected via a power cable leading into the housing;

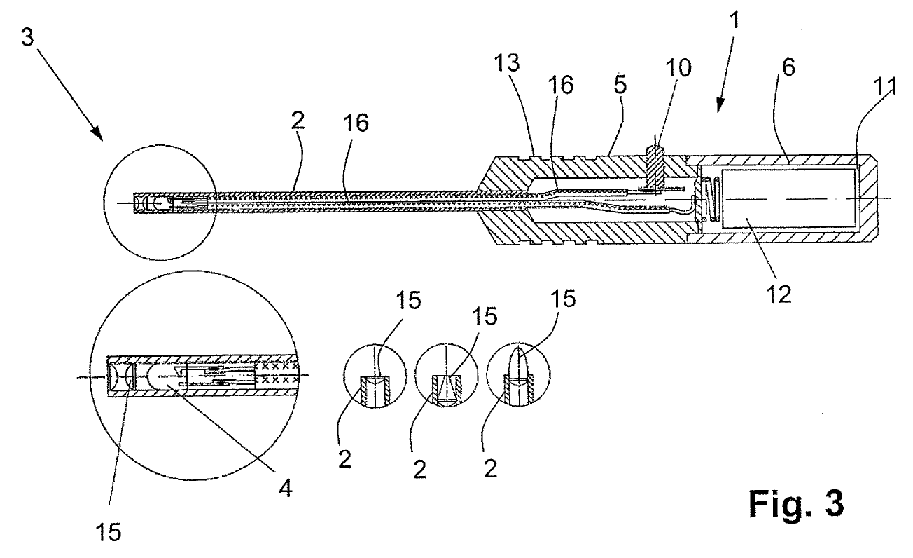

FIG. 3 shows in a schematic view, in section, a third exemplary embodiment of a handheld instrument according to the invention, the light source being integrated into the probe and the energy source being integrated into the housing;

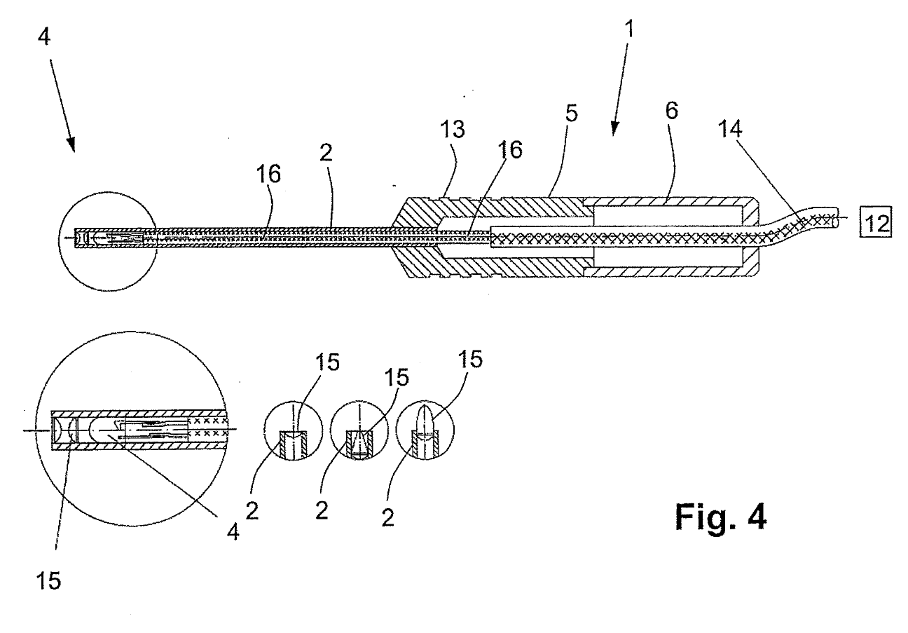

FIG. 4 shows in a schematic view, in section, fourth exemplary embodiment of a handheld instrument according to the invention, in which the light source is integrated into the probe and the energy supply is effected via a power cable leading into the housing.

FIGS. 1 to 4 show four different exemplary embodiments of a handheld medical instrument serving for illumination, this handheld instrument being suitable in particular for application in ophthalmology. The handheld instrument comprises a housing 1 serving as a handle, and a probe 2 extending from the housing 1 and having a light outlet 3 at the end. The probe 2 is suitable for being introduced into body openings or for illumination on or in the eye.

In a manner according to the invention, the handheld instrument comprises an integrated light source 4, which in concrete terms—in all four exemplary embodiments—is a light emitting diode (LED).

In the exemplary embodiment shown in FIG. 1, the housing 1 comprises two housing halves 5, 6, the front housing half 5 carrying the probe 2. Furthermore, the light source 4 is arranged in the front housing half 5, the emitted light from said light source coupling through a lens arrangement 7 into an optical fiber 8 extending through the probe 2.

In order to avoid accumulation of heat within the front housing half 5, the inner wall of the housing half 5 is equipped with a type of cooling fins 9 for heat dissipation. The material of the front housing half 5 correspondingly has a high specific thermal conductivity.

FIG. 1 furthermore reveals that a switch 10 is provided in the front housing half 5, said switch serving for switching the light source 4 on and off.

A battery compartment 11 is provided in the rear housing half 6, a battery 12—which is only indicated in the figures—or a corresponding rechargeable battery being arranged in said compartment. Consequently, the variant of a handheld instrument according to the invention as shown in FIG. 1 comprises both an integrated light source 4 and an integrated energy source 12, which is only represented symbolically within the battery compartment 11.

FIG. 1 furthermore reveals that the housing 1 serving as a handle, in concrete terms the front housing half 5, is equipped with a particular profiling 13 on the outer surface in order to increase the reliability of gripping the handheld instrument.

In the exemplary embodiment shown in FIG. 1, the probe 2 is embodied in rectilinear fashion. The optical fiber 8 extends within the probe 2 and conducts the light from the light source 4, said light being coupled in in the front housing half 5, to the light outlet 3.

The situation in the region of the light outlet 3 can be configured in different ways, as is illustrated by three variants in FIG. 1. It is thus possible, for example, for the optical fiber 8 to end flush with the probe 2. Likewise, the optical fiber 8 can taper or be rounded in the front region. It can end within the probe 2 or alternatively in slightly exposed fashion, i.e. in a manner projecting from the probe 2. Different illumination situations can be generated from the concrete arrangement or configuration of the optical fiber 8 in the region of the light outlet 3.

The exemplary embodiment shown in FIG. 2 differs from the exemplary embodiment from FIG. 1 only by virtue of the fact that in the former the energy source 12 is arranged outside the handheld instrument, that is to say outside the housing 1. The power supply is effected via a power cable 14, which is electrically connected to the terminals of the light source 4 in the interior of the housing 1. Otherwise, the exemplary embodiment shown in FIG. 2 corresponds to the exemplary embodiment from FIG. 1.

FIG. 3 shows a third exemplary embodiment of a handheld instrument according to the invention, in which the light source 4 is arranged in the region at the end of the probe 2. The energy source 12 is situated in the battery compartment 11, such that here both the light source 4 and the energy source 12 are integrated.

The light source 4 is a miniaturized LED, adjacent to which is a miniaturized lens arrangement 15. Said lens arrangement 15 can end flush with the probe 2. It is likewise conceivable for the lens arrangement 15 to end in the probe 2 or to project from the probe 2. Different forms of the lens arrangement 15, in particular of the respective outer lens, are conceivable in accordance with the illustration in FIG. 3.

A power cable 16 extends through the probe 2, and is used to supply power to the miniaturized light source 4 in the probe 2.

FIG. 4 shows a fourth exemplary embodiment of a handheld instrument according to the invention, in which the energy source 12 is arranged externally. The electrical power is passed into the housing 1 via a power cable 14, power being supplied to the light source 4 situated in the probe 2 via the further power cable 16. The two power cables 14 and 16 can be one and the same lead. The same explanations as for FIG. 3 are applicable with regard to the lens arrangement 15 at the end.

Finally, it should be noted that the exemplary embodiments discussed above serve only for discussing the handheld medical instrument according to the invention by way of example, but they do not restrict said instrument to the exemplary embodiments.

1-42. (canceled)

43. A handheld ophthalmology instrument for illumination comprising:
a housing serving as a handle;
a probe extending from the housing, and a light outlet at the end; and
a light source integrated into the handheld instrument.

44. The handheld instrument as claimed in claim 43, wherein the light source is surrounded by a material suitable for heat dissipation or by a heat conducting device.

45. The handheld instrument as claimed in claim 43, wherein the light source is arranged in the housing, wherein the handheld instrument further comprises an optical fiber extending through the probe, and wherein light emitted by the light source passes to the light outlet via the optical fiber.

46. The handheld instrument as claimed in claim 45, wherein the intensity of the light emitted by the light source or the intensity of the light that can be coupled into the optical fiber is adjustable.

47. The handheld instrument as claimed in claim 45, wherein the handheld instrument further comprises optical components arranged between the light source and the light outlet and wherein the light from the light source can be coupled into the optical fiber via the optical components.

48. The handheld instrument as claimed in claim 47, wherein the optical components comprise a lens, an arrangement of lenses, a filter or an arrangement of filters.

49. The handheld instrument as claimed in claim 45, wherein the optical fiber ends flush with the free end of the probe, ends within the probe just before the free end thereof, or ends with a flat or rounded end within or outside the probe.

50. The handheld instrument as claimed in claim 45, wherein the optical fiber is displaceable from a position with the free end within the probe into a position with the free end at least slightly outside the probe.

51. The handheld instrument as claimed in claim 50, wherein the displaceability of the optical fiber is realized by at least one of a relative movement of the probe with respect to the housing or by means of an actuating member that is assigned to the handle and acts directly or indirectly on the optical fiber.

52. The handheld instrument as claimed in claim 43, wherein the handheld instrument comprises an optical assembly in the form of a miniaturized lens or a miniaturized arrangement of lenses, and wherein the optical assembly is assigned directly to the light outlet.

53. The handheld instrument as claimed in claim 52, wherein the optical assembly ends flush with the free end of the probe, ends within the probe just before the free end thereof, or ends with a flat or rounded end within or outside the probe.

54. The handheld instrument as claimed in claim 53, wherein the optical assembly is displaceable from a position within the probe into a position at least slightly outside the probe.

55. The handheld instrument as claimed in claim 54, wherein the displaceability of the optical assembly is effected by an actuating member assigned to the probe or to the housing.

56. The handheld instrument as claimed in claim 43, wherein the light source comprises at least one light emitting diode (LED).

57. The handheld instrument as claimed in claim 56, wherein the light source comprises at least one of two or more light emitting diodes for emitting light of different wavelengths or two or more light emitting diodes that can optionally be switched individually or jointly.

58. The handheld instrument as claimed in claim 43, wherein the handheld instrument further comprises at least one of an external energy source connected to the light source via a power cable leading into the housing or an internal energy source in the form of a battery or a rechargeable battery connected to the light source.

59. The handheld instrument as claimed in claim 43, wherein the housing is embodied in two parts and can preferably be opened on the side remote from the light outlet.

60. The handheld instrument as claimed in claim 58 wherein the light source together with the internal energy source form a monolithic assembly that can be removed from the housing or can be removed with a part of the housing.

61. The handheld instrument as claimed in claim 43, wherein the light source is structured to be removed and replaced with light sources having different wavelengths and/or different light intensities.

62. The handheld instrument as claimed in claim 43, wherein the handheld instrument comprises an external optical fiber and wherein the housing comprises a coupling location for coupling out light by means of the external optical fiber that can be coupled thereto.
